SEQUENCE LISTING (11 sequences)

SEQ ID NO: 1 - DNA, 2325 bp, Dengue virus type 1
atgaacaaccaacggaaaaagacgggtcgaccgtctttcaatatgctgaaacgcgcgagaaaccgcgtgtcaactgtttcacagttggcgaagagattctcaaaaggattgctttcaggccaaggacccatgaaattggtgatggcttttatggcattcctaagatttctagccatacccccaacagcaggaattttggctagatggggctcactcaagaagaatggagcgattaaagtgctacggggtttcaagaaagaaatctcaaacatgctgagcataatgaatagaagaaaaagatctgtgaccatgctccttatgctgctgcccacagccctggcgttccatctgaccacacgagggggagagccgcacatgatagttagcaagcaggaaagaggaaagtcacttttgtttaagacctctgcaggtgtcaacatgtgcaccattattgcgatggatttgggagagttatgtgaggacacaatgacctacaaatgcccccggatcactaaggcggaaccagatgacgttgactgttggtgcaatgccacggacacatgggtgacctatggaacgtgttctcgaactggcgaacaccgacgagacaagcgttccgtcgcactggccccacatgtggggcttggtctagaaacaagagccgaaacgtggatgtcctctgaaggcgcttggaaacaaatacaaaaagtggagacttgggctctgagacacccaggattcacggtaatagccctctttctagcacatgccataggaacatccatcacccagaaagggattattttcattttgttgatgctggtaacaccatccatggccatgcgatgcgtgggaataggcaacagagacttcgtggaaggactgtcaggagcaacgtgggtggatgtggtactggagcatggaagttgcgtcaccaccatggcaaaagataaaccaacattggacattgaactcttgaagacggaggtcacaaagcctgccgtcctgcgtaaactgtgcattgaagctaaaatatcaaacaccaccaccgattcaagatgtccaacacaaggggaagccacactggtggaagaacaagacgcgaacttcgtgtgtcgacgaacgtttgtggacagaggctggggcaatggctgtgggcttttcggaaaaggtagcctaataacgtgtgctaagttcaagtgtgtgacaaaactggaaggaaagattgttcaatatgagaacttgaaatattcagtgatagtcaccgtccacactggtgaccagcaccaggtgggaaatgagaccacagaacatggaacaattgcaaccataacacctcaagctcctacgtcggaaatacagctgaccgactacggagctcttacattggattgctcacccagaacagggctagactttaatgagatggtgttgttgacaatgaaagaaaaatcatggcttgtccacaaacaatggtttctagacttaccactgccctggacctcgggagcttcaacaccccaagagacttggaacagagaagatttgctggttacatttaagacagctcatgcaaagaagcaggaagtagtcgtactaggatcacaagaaggagcaatgcacactgcgttgaccggagcgacagaaatccaaacgtctggaacgacaaaaatttttgcaggacacttgaaatgtagactaaaaatgaacaaactgaccttaaaagggatgtcatatgtgatgtgcacaggttcattcaagttagagaaagaagtggctgagacccagcatggaactgttctagtgcaggttaaatacgaaggaacagatgcaccatgcaagatccccttttcgacccaagatgagaaaggagtaacccagaatgggagattgataacagccaaccccatagtcactgacaaagaaaaaccagtcaacattgaggcagaaccaccttttggtgagagttacatcgtggtaggagcaggtgaaaaagctttgaaattaagctggttcaagaaaggaagcagcatagggaaaatgcttgaagcaactgcccgaggagcacgaaggatggccatcctaggagacaccgcatgggacttcggttctataggaggagtgttcacgtctgtgggaaaactggtacaccagatctttggaactgcatatggagttttgttcagcggtgtttcctggactatgaaaataggaatagggattctgctgacatggctaggattaaattcaaggagcgcgtccctttcgatgacgtgcattgcagttggcatggttacactgtacctaggagtcatggttcaggcg

SEQ ID NO: 2 - DNA, 2025 bp, Dengue virus type 1
tctgtgaccatgctccttatgctgctgcccacagccctggcgttccatctgaccacacgagggggagagccgcacatgatagttagcaagcaggaaagaggaaagtcacttttgtttaagacctctgcaggtgtcaacatgtgcaccattattgcgatggatttgggagagttatgtgaggacacaatgacctacaaatgcccccggatcactaaggcggaaccagatgacgttgactgttggtgcaatgccacggacacatgggtgacctatggaacgtgttctcgaactggcgaacaccgacgagacaagcgttccgtcgcactggccccacatgtggggcttggtctagaaacaagagccgaaacgtggatgtcctctgaaggcgcttggaaacaaatacaaaaagtggagacttgggctctgagacacccaggattcacggtaatagccctctttctagcacatgccataggaacatccatcacccagaaagggattattttcattttgttgatgctggtaacaccatccatggccatgcgatgcgtgggaataggcaacagagacttcgtggaaggactgtcaggagcaacgtgggtggatgtggtactggagcatggaagttgcgtcaccaccatggcaaaagataaaccaacattggacattgaactcttgaagacggaggtcacaaagcctgccgtcctgcgtaaactgtgcattgaagctaaaatatcaaacaccaccaccgattcaagatgtccaacacaaggggaagccacactggtggaagaacaagacgcgaacttcgtgtgtcgacgaacgtttgtggacagaggctggggcaatggctgtgggcttttcggaaaaggtagcctaataacgtgtgctaagttcaagtgtgtgacaaaactggaaggaaagattgttcaatatgagaacttgaaatattcagtgatagtcaccgtccacactggtgaccagcaccaggtgggaaatgagaccacagaacatggaacaattgcaaccataacacctcaagctcctacgtcggaaatacagctgaccgactacggagctcttacattggattgctcacccagaacagggctagactttaatgagatggtgttgttgacaatgaaagaaaaatcatggcttgtccacaaacaatggtttctagacttaccactgccctggacctcgggagcttcaacaccccaagagacttggaacagagaagatttgctggttacatttaagacagctcatgcaaagaagcaggaagtagtcgtactaggatcacaagaaggagcaatgcacactgcgttgaccggagcgacagaaatccaaacgtctggaacgacaaaaatttttgcaggacacttgaaatgtagactaaaaatgaacaaactgaccttaaaagggatgtcatatgtgatgtgcacaggttcattcaagttagagaaagaagtggctgagacccagcatggaactgttctagtgcaggttaaatacgaaggaacagatgcaccatgcaagatccccttttcgacccaagatgagaaaggagtaacccagaatgggagattgataacagccaaccccatagtcactgacaaagaaaaaccagtcaacattgaggcagaaccaccttttggtgagagttacatcgtggtaggagcaggtgaaaaagctttgaaattaagctggttcaagaaaggaagcagcatagggaaaatgcttgaagcaactgcccgaggagcacgaaggatggccatcctaggagacaccgcatgggacttcggttctataggaggagtgttcacgtctgtgggaaaactggtacaccagatctttggaactgcatatggagttttgttcagcggtgtttcctggactatgaaaataggaatagggattctgctgacatggctaggattaaattcaaggagcgcgtccctttcgatgacgtgcattgcagttggcatggttacactgtacctaggagtcatggttcaggcg

SEQ ID NO: 3 - DNA, 300 bp, Dengue virus type 1
atgaacaaccaacggaaaaagacgggtcgaccgtctttcaatatgctgaaacgcgcgagaaaccgcgtgtcaactgtttcacagttggcgaagagattctcaaaaggattgctttcaggccaaggacccatgaaattggtgatggcttttatggcattcctaagatttctagccatacccccaacagcaggaattttggctagatggggctcactcaagaagaatggagcgattaaagtgctacggggtttcaagaaagaaatctcaaacatgctgagcataatgaatagaagaaaaaga

SEQ ID NO: 4 - DNA, 1485 bp, Dengue virus type 1
atgcgatgcgtgggaataggcaacagagacttcgtggaaggactgtcaggagcaacgtgggtggatgtggtactggagcatggaagttgcgtcaccaccatggcaaaagataaaccaacattggacattgaactcttgaagacggaggtcacaaagcctgccgtcctgcgtaaactgtgcattgaagctaaaatatcaaacaccaccaccgattcaagatgtccaacacaaggggaagccacactggtggaagaacaagacgcgaacttcgtgtgtcgacgaacgtttgtggacagaggctggggcaatggctgtgggcttttcggaaaaggtagcctaataacgtgtgctaagttcaagtgtgtgacaaaactggaaggaaagattgttcaatatgagaacttgaaatattcagtgatagtcaccgtccacactggtgaccagcaccaggtgggaaatgagaccacagaacatggaacaattgcaaccataacacctcaagctcctacgtcggaaatacagctgaccgactacggagctcttacattggattgctcacccagaacagggctagactttaatgagatggtgttgttgacaatgaaagaaaaatcatggcttgtccacaaacaatggtttctagacttaccactgccctggacctcgggagcttcaacaccccaagagacttggaacagagaagatttgctggttacatttaagacagctcatgcaaagaagcaggaagtagtcgtactaggatcacaagaaggagcaatgcacactgcgttgaccggagcgacagaaatccaaacgtctggaacgacaaaaatttttgcaggacacttgaaatgtagactaaaaatgaacaaactgaccttaaaagggatgtcatatgtgatgtgcacaggttcattcaagttagagaaagaagtggctgagacccagcatggaactgttctagtgcaggttaaatacgaaggaacagatgcaccatgcaagatccccttttcgacccaagatgagaaaggagtaacccagaatgggagattgataacagccaaccccatagtcactgacaaagaaaaaccagtcaacattgaggcagaaccaccttttggtgagagttacatcgtggtaggagcaggtgaaaaagctttgaaattaagctggttcaagaaaggaagcagcatagggaaaatgcttgaagcaactgcccgaggagcacgaaggatggccatcctaggagacaccgcatgggacttcggttctataggaggagtgttcacgtctgtgggaaaactggtacaccagatctttggaactgcatatggagttttgttcagcggtgtttcctggactatgaaaataggaatagggattctgctgacatggctaggattaaattcaaggagcgcgtccctttcgatgacgtgcattgcagttggcatggttacactgtacctaggagtcatggttcaggcg

SEQ ID NO: 5 - DNA, 2321 bp, Dengue virus type 2
atgaccaacggaaaaaggcgaaaaacacgccattcaatatgctgaaacgcgagagaaaccgcgtgtcgactgtgcaacagctgacaaagagattctcacttggaatgctacagggacgaggaccactaaaactgtacatggccctggtggcgttccttcgtttcctaacaatcccaccaacagcagggatattgaagagatggggaacaattaaaaaatccaaagctattaatgttttgagagggttcaggaaagagattggaaggatgctgaacatcttgaataggagacgcagatctgcaggcatgatcattatgctgattccaacagtgatggcgttccatttaaccacacgtaacggagaaccacacatgatcgtcagcagacaagagaaagggaaaagtcttctgtttaaaacagaggatggcgtgaacatgtgtaccctcatggccatggaccttggtgaattgtgtgaagacacaatcacgtacaagtgtccccttctcaggcagaatgagccagaagacatagactgttggtgcaactctacctccacgtgggtaacttatgggacgtgtaccaccatgggagaacatagaagacaaaaaagatcagtggcactcgttccacatgtgggaatgggactggagacacgaactgaaacatggatgtcatcagaaggggcctggaaacatgtccagagaattgaaacttggatcttgagacatccaggcttcaccatgatggcagcaatcctggcatacaccataggaacgacacatttccaaagagccctgattttcatcttactgacagctgtcactccttcaatgacaatgcgttgcataggaatgtcaaatagagactttgtggaaggggtttcaggaggaagctgggttgacatagtcttagaacatggaagctgtgtgacgacgatggcaaaaaacaaaccaacattggattttgaactgataaaaacagaagccaaacagcctgccaccctaaggaagtactgtatagaggcaaagctaaccaacacaacaacagaatctcgctgcccaacacaaggggaacccagcctaaatgaagagcaggacaaaaggttcgtctgcaaacactccatggtagacagaggatggggaaatggatgtggactatttggaaagggaggcattgtgacctgtgctatgttcagatgcaaaaagaacatggaaggaaaagttgtgcaaccagaaaacttggaatacaccattgtgataacacctcactcaggggaagagcatgcagtcggaaatgacacaggaaaacatggcaaggaaatcaaaataacaccacagagttccaccacagaagcagaattgacaggttatggcactgtcacaatggagtgctctccaagaacgggcctcgacttcaatgagatggtgttgctgcagatggaaaataaagcttggctggtgcacaggcaatggttcctagacctgccgttaccatggttgcccggagcggacacacaagggtcaaattggatacagaaagagacattggtcactttcaaaaatccccatgcgaagaaacaggatgttgttgttttaggatcccaagaaggggccatgcacacagcacttacaggggccacagaaatccaaatgtcatcaggaaacttactcttcacaggacatctcaagtgcaggctgagaatggacaagctacagctcaaaggaatgtcatactctatgtgcacaggaaagtttaaagttgtgaaggaaatagcagaaacacaacatggaacaatagttatcagagtgcaatatgaaggggacggctctccatgcaagatcccttttgagataatggatttggaaaaaagacatgtcttaggtcgcctgattacagtcaacccaattgtgacagaaaaagatagcccagtcaacatagaagcagaacctccattcggagacagctacatcatcataggagtagagccgggacaactgaagctcaactggtttaagaaaggaagttctatcggccaaatgtttgagacaacaatgaggggggcgaagagaatggccattttaggtgacacagcctgggattttggatccttgggaggagtgtttacatctataggaaaggctctccaccaagtctttggagcaatctatggagctgccttcagtggggtttcatggactatgaaaatcctcataggagtcattatcacatggataggaatgaattcacgcagcacctcactgtctgtgacactagtattggtgggaattgtgacactgtatttgggagtcatggtgcaggcc

SEQ ID NO: 6 - DNA, 2319 bp, Dengue virus type 3
atgaacaaccaacggaaaaagacgggaaaaccgtctatcaatatgctgaaacgcgtgagaaaccgtgtgtcaactggatcacagttggcgaagagattctcaagaggattgctgaacggccaaggaccaatgaaattggttatggcgtttatagctttcctcagatttctagccattccaccgacagcaggagtcttggctagatggggtacctttaagaagtcgggggctattaaggtcttaaaaggcttcaagaaggagatctcaaacatgctgagcattatcaacaaacggaaaaagacatcgctctgtctcatgatgatgttaccagcaacacttgctttccacttaacttcacgagatggagagccgcgcatgattgtggggaagaatgaaagaggaaaatccctactttttaagacagcctctggaatcaacatgtgcacactcatagccatggatttgggagagatgtgtgatgacacggtcacttacaaatgcccccacattaccgaagtggagcctgaagacattgactgttggtgcaaccttacatcgacatgggtgacttatggaacatgcaatcaagctggagagcatagacgcgataagagatcagtggcgttagctccccatgtcggcatgggactggacacacgcactcaaacctggatgtcggctgaaggagcttggagacaagtcgagaaggtagagacatgggcccttaggcacccagggtttaccatactagccctatttcttgcccattacataggcacttccttgacccagaaagtggttatttttatactattaatgctggttaccccatccatgacaatgagatgtgtgggagtaggaaacagagattttgtggaaggcctatcgggagctacgtgggttgacgtggtgctcgagcacggtgggtgtgtgactaccatggctaagaacaagcccacgctggacatagagcttcagaagactgaggccactcagctggcgaccctaaggaagctatgcattgagggaaaaattaccaacataacaaccgactcaagatgtcccacccaaggggaagcgattttacctgaggagcaggaccagaactacgtgtgtaagcatacatacgtggacagaggctggggaaacggttgtggtttgtttggcaagggaagcttggtgacatgcgcgaaatttcaatgtttagaatcaatagagggaaaagtggtgcaacatgagaacctcaaatacaccgtcatcatcacagtgcacacaggagaccaacaccaggtgggaaatgaaacgcagggagttacggctgagataacatcccaggcatcaaccgctgaagccattttacctgaatatggaaccctcgggctagaatgctcaccacggacaggtttggatttcaatgaaatgattttattgacaatgaagaacaaagcatggatggtacatagacaatggttctttgacttacccctaccatggacatcaggagctacaacaaaaacaccaacttggaacaggaaagagcttcttgtgacatttaaaaatgcacatgcaaaaaagcaagaagtagttgtccttggatcacaagagggagcaatgcatacagcactgacaggagctacagagatccaaacctcaggaggcacaagtatttttgcggggcacttaaaatgtagactcaagatggacaaattgaaactcaaggggatgagctatgcaatgtgcttgaatacctttgtgttgaagaaagaagtctccgaaacgcagcatgggacaatactcattaaggttgagtacaaaggggaagatgcaccctgcaagattcctttctccacggaggatggacaagggaaagctcacaatggcagactgatcacagccaatccagtggtgaccaagaaggaggagcctgtcaacattgaggctgaacctccttttggggaaagtaatatagtaattggaattggagacaaagccctgaaaatcaactggtacaggaagggaagctcgattgggaagatgttcgaggccactgccagaggtgcaaggcgcatggccatcttgggagacacagcctgggactttggatcagtgggtggtgttttgaattcattagggaaaatggtccaccaaatatttgggagtgcttacacagccctatttagtggagtctcctggataatgaaaattggaataggtgtcctcttaacctggatagggttgaattcaaaaaacacttctatgtcattttcatgcattgcgataggaatcattacactctatctgggggtcgtggtgcaagct

SEQ ID NO: 7 - DNA, 2319 bp, Dengue virus type 4
atgaaccaacgaaaaaaggtggttagaccacctttcaatatgctgaaacgcgagagaaaccgcgtatcaacccctcaagggttggtgaagagattctcaaccggacttttttccgggaaaggacccttacggatggtgctagcattcatcacgtttttgcgagtcctttccatcccaccaacagcagggattctgaagagatggggacagttgaagaaaaacaaggccatcaaaatactgactggattcaggaaggagataggccgcatgctgaacatcttgaatgggagaaaaaggtcaacaatgacattgctgtgcttgattcccaccgtaatggcgtttcacttgtcaacaagagatggcgaaccccttatgatagtggcaaaacacgaaagggggagacctctcttgtttaagacaacagagggaatcaacaaatgcactcttattgccatggacctgggtgaaatgtgtgaggacaccgtcacgtatgagtgccctctactggtcaataccgaacctgaggacattgattgctggtgcaatctcacgtctgcgtgggtcatgtatgggacatgcactcagagtggggaacggagacgggagaagcgctcagtagccctaacaccacattcaggaatgggattggagacaagggctgagacatggatgtcatcggaaggggcttggaaacatgctcagagggtagagagttggatactcagaaacccaggattcgctctcttggcaggatttatggcctatatgattgggcaaacaggaatccagcgaacagtcttctttgttctaatgatgctggtcgccccatcctacggaatgcgatgcgtgggagtggggaacagagactttgtggaaggagtctcaggtggagcatgggtcgattcggtgctagaacatggaggatgtgtcacaaccatggcccagggaaaaccaaccttggattttgaactgatcaagacaacagccaaggaagtggctctgttaagaacctattgcattgaagcctcgatatcaaacataaccacggcaacaagatgtccaacgcaaggagaaccttatctcaaagaggaacaagatcaacagtacatttgccggagagatgtggtagacagagggtggggcaatggctgtggcttgtttgggaaaggaggagttgtgacatgtgcgaagttttcatgctcggggaagataacaggcaatttggtccaaattgagaaccttgaatacacagtgattgtaacagtccacaatggagacacccatgcagtaggaaatgacacatccaaccatggagtgacagccacgataacccccaggtcaccatcggtagaagttaaattaccggattatggagaattaacactcgattgtgaacccaggtccggaattgattttaatgagatgattctgatgaaaatgaaaaagaaaacgtggcttgtgcacaagcaatggtttttggatctacctctaccatgggcagcaggagcagacacatcagaagttcattggaattacaaagagagaatggtgacattcaaggttcctcatgccaagagacaggatgtgatagtgctaggatctcaggaaggagccatgcattctgccctcaccggagctacagaagtggattccggtgatggaaaccacatgtttgcaggacatcttaaatgcaaagttcgcatggagaaattgagaattaagggaatgtcatacacgatgtgctcaggaaagttctcaattgacaaagagatggcagaaacacagcatgggacaacagtggtaaaagtcaagtatgagggtgctggagctccatgtaaagttcccatagagataagagatgtgaacaaggaaaaagtggtagggcgtatcatctcacctaccccttttgctgagaataccaacagtgtaaccaacatagaattagaacgccctttggacagctacatagtaataggtgttggagacagcgcattaacactccattggttcaggaaagggagttccattggcaagatgtttgagtccacatacagaggcgcaaagcgaatggccattctaggtgaaacagcctgggattttggttctgttggtggactgctcacatcattgggaaaggctgtacaccaggtttttggtagtgtgtatacaactatgtttggaggagtctcatggatggttagaatcctaattgggttcttagtgttgtggattggcacgaactcgaggaacacttcaatggctatgacgtgcatagctgttggaggaatcactctgtttctgggcttcacagttcaagca

SEQ ID NO: 8 - DNA, 57 bp, Foot-and-mouth disease virus
agtaacttcgacctcctcaagttggcgggagacgttgagtccaaccccggacccgcc

SEQ ID NO: 9 - DNA, 2373 bp, West Nile virus
atgtctaagaaaccaggagggcccggcaagagccgggctgtcaatatgctaaaacgcggaatgccccgcgtgttgtccttgattggactgaagagggctatgttgagcctgatcgacggcaaggggccaatacgatttgtgttggctctcttggcgttcttcaggttcacagcaattgctccgacccgagcagtgctggatcgatggagaggtgtgaacaaacaaacagcgatgaaacaccttctgagttttaagaaggaactagggaccttgaccagtgctatcaatcggcggagctcaaaacaaaagaaaagaggaggaaagaccggaattgcagtcatgattggcctgatcgccagcgtaggagcagttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtactgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagcttcaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccgaccatgggagaagctcacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccatcttgaaagagaatatcaagtacgaagtggccatttttgtccatggaccaactactgtggagtcgcacggaaactactccacacaggttggagccactcaggcagggagactcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccattggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgataggtccatagctctcacgtttctcgcagttggaggagttctgctcttcctctccgtgaacgtgcacgct

SEQ ID NO: 10 - DNA, 2058 bp, West Nile virus
ggaggaaagaccggaattgcagtcatgattggcctgatcgccagcgtaggagcagttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtactgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagcttcaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccgaccatgggagaagctcacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccatcttgaaagagaatatcaagtacgaagtggccatttttgtccatggaccaactactgtggagtcgcacggaaactactccacacaggttggagccactcaggcagggagactcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccattggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgataggtccatagctctcacgtttctcgcagttggaggagttctgctcttcctctccgtgaacgtgcacgct

SEQ ID NO: 11 - DNA, 315 bp, West Nile virus
atgtctaagaaaccaggagggcccggcaagagccgggctgtcaatatgctaaaacgcggaatgccccgcgtgttgtccttgattggactgaagagggctatgttgagcctgatcgacggcaaggggccaatacgatttgtgttggctctcttggcgttcttcaggttcacagcaattgctccgacccgagcagtgctggatcgatggagaggtgtgaacaaacaaacagcgatgaaacaccttctgagttttaagaaggaactagggaccttgaccagtgctatcaatcggcggagctcaaaacaaaagaaaaga

(19) United States
(12) Patent Application Publication
(10) Pub. No.: US 2014/0220061 A1
(43) Pub. Date: Aug. 7, 2014

(54) YEAST EXPRESSION OF FLAVIVIRUS VIRUS-LIKE PARTICLES AND USE THEREOF

(71) Applicants: Jared D. Evans, Pittsburgh, PA (US); Nicole M. Beatty, Pittsburgh, PA (US)

(72) Inventors: Jared D. Evans, Pittsburgh, PA (US); Nicole M. Beatty, Pittsburgh, PA (US)

(73) Assignee: University of Pittsburgh - Of the Commonwealth System of Higher Education, Pittsburgh, PA (US)

(21) Appl. No.: 14/241,530

(22) PCT Filed: Aug. 30, 2012
(86) PCT No.: PCT/US2012/053090

§ 371 Date: Feb. 27, 2014

Related U.S. Application Data
(60) Provisional application No. 61/529,061, filed on Aug. 30, 2011.

(51) Int. Cl. C12N 7/00

(52) U.S. Cl. CPC C12N 7/00
USPC 424/186.1; 435/471; 435/320.1; 435/254.2; 435/235.1

(57) ABSTRACT

Described herein is a method for the production of flavivirus virus-like particles (VLPs) in a yeast system. In some cases, flavivirus structural proteins are expressed in a Pichia pastoris strain and isolated using pressurized mechanical lysis. The disclosed VLPs can be used as immunogenic compositions for the prevention or treatment of flavivirus infection. Also described are isolated nucleic acid molecules encoding a flavivirus capsid (C) protein; a flavivirus premembrane (prM) protein; and at least one flavivirus envelope (E) protein or a flavivirus E protein lacking the transmembrane domain (EΔTM), wherein the nucleic acid molecule further encodes at least one foot and mouth disease virus (FMDV) 2A autocatalytic site (2A) positioned between two flavivirus protein coding sequences. Expression of such nucleic acid molecules in a host cell produces flavivirus VLPs.

### CROSS REFERENCE TO RELATED APPLICATIONS

This application claims the benefit of U.S. Provisional Application No. 61/529,061, filed Aug. 30, 2011, which is herein incorporated by reference in its entirety.

### FIELD

This disclosure concerns the production of flavivirus virus-like particles (VLPs) by expression of flavivirus structural proteins in yeast, such a Pichia pastoris. The present disclosure further concerns use of the flavivirus VLPs as immunogenic compositions.

### BACKGROUND

Flaviviruses are significant human pathogens for which no commercially approved vaccines exist. Flaviviruses exist as small (50 nm) icosahedral particles containing a single RNA molecule encoding 3 structural proteins (C, M and E) that make up the virion, and 7 nonstructural proteins required for genome replication. This virus family includes a number of mosquito-borne viruses that are pathogenic for humans, including West Nile virus (WNV), dengue virus (DENV), Japanese encephalitis virus (JEV), and yellow fever virus (YFV). Each virus is endemic in regions with a large and highly susceptible population, causing significant medical and economic burden.

DENV and WNV are closely related flaviviruses that cause severe human disease as well as heavy medical and economic burden. Outcomes of WNV infection range from benign, non-symptomatic infection to flu-like febrile illness to severe central nervous system (CNS) manifestations. Neurological sequelae of infection include disorientation/confusion, flaccid paralysis, and meningo-encephalitis. Importantly, the neuropathogenesis of WNV has increased during its global expansion, becoming the major cause of viral encephalitis in the Western hemisphere, DENV has four known serotypes (1-4) that have defined global distribution. However, modern travel has altered the pattern and introduced DENV into naive populations. Over the past 50 years, dengue virus has become the most significant arbovirus human pathogen in the world because of its unusual transmission cycle involving a human host for amplification. There are over 50 million cases recorded each year with 2.5 billion people at risk. Recent outbreaks in India, Brazil, and United States highlight the importance of developing new defense and treatment options for this emerging infectious disease.

### SUMMARY

Disclosed herein is a method for the production of flavivirus virus-like particles (VLPs) in a yeast system. In some cases, flavivirus structural proteins are expressed in yeast, such as in a Pichia pastoris strain, and isolated using pressurized mechanical lysis. The disclosed VLPs can be used as immunogenic compositions for the prevention or treatment of flavivirus infection.

Provided herein are isolated nucleic acid molecules encoding a flavivirus capsid (C) protein; a flavivirus premembrane (prM) protein; and at least one flavivirus envelope (E) protein or a flavivirus E protein lacking the transmembrane domain (EΔTM), wherein the nucleic acid molecule further encodes at least one foot and mouth disease virus (FMDV) 2A autocatalytic site (2A) positioned between two flavivirus protein coding sequences. Expression of the nucleic acid molecules in a host cell produces flavivirus VLPs. Also provided are vectors comprising the disclosed nucleic acid molecules and host cells containing the vectors.

Further provided is a method of producing flavivirus VLPs. In some embodiments, the method includes transforming yeast cells with a nucleic acid molecule encoding a flavivirus premembrane (prM) protein; and at least one flavivirus envelope (E) protein or a flavivirus E protein lacking the transmembrane domain (EΔTM); cultivating the transformed yeast cells under conditions sufficient to allow for expression of the flavivirus proteins and formation of VLPs; subjecting the yeast cells to pressurized mechanical lysis; and isolating the VLPs from the yeast cell lysis.

Compositions comprising the VLPs disclosed herein are also provided by the present disclosure. Also provided are methods of eliciting an immune response in a subject against flavivirus by administering to the subject a VLP (or composition thereof) as disclosed herein.

The foregoing and other features and advantages will become more apparent from the following detailed description of several embodiments, which proceeds with reference to the accompanying figures.

### BRIEF DESCRIPTION OF THE FIGURES

FIGS. 1A and 1B: Cloning of WNV structural genes and integration into Pichia pastoris. (A) Schematic of WNV structural genes cloned into plasmid pPIC3.5. WNV C-prM-E genetic sequences were RT-PCR amplified from WNV strain NY99 viral RNA and cloned into pPIC3.5 transfer vector (Invitrogen). (B) PCR identification of Pichia colonies with integrated expression cassette. Lane 1: Marker; Lane 2: WNV C-prM-E-PIC3.5 plasmid (positive control); Lane 3: pPIC3.5 (negative control); Lanes 4-11: Eight Pichia colonies from selection plates. Black arrow denotes colony with integrated C-prM-E cassette. Empty arrow denotes false positive.

FIGS. 2A and 2B: Expression of WNV C-prM-E in Pichia pastoris. P. pastoris containing WNV C-prM-E cassette (lanes 4-7) or parental yeast (lanes 9-12) were induced for 0 (lanes 4 and 9), 24 (lanes 5 and 10), 48 (lanes 6 and 11), or 72 (lanes 7 and 12) hours. Total protein (15 μg) was separated on SDS-PAGE. Protein expression was analyzed by Coomassie stain (A) and Western blot (B) with anti-WNV antibodies. Lane 1: protein marker; Lane 2: mock-infected Vero cell lysate (negative control); Lane 3: WNV-infected Vero cell lysate (positive control).

FIGS. 3A and 3B: Yeast-produced VLPs sediment at similar density as virus particles. (A) Western blot of yeast-produced DENV-1 VLPs and DENV-1 infectious virus from infected Vero cell lysates (lane 2) or sucrose gradient fractions 1-12 (lanes 3-14). (B) Western blot of yeast-produced WNV VLPs from sucrose gradient fractions 1-11 (lanes 2-12) and post-immunoprecipitation supernatant (lane 13). Lane 14 shows WNV from infected Vero cell lysates.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created Aug. 20, 2012, 24.2 KB, which is incorporated by reference herein. In the accompanying sequence listing:

SEQ ID NO: 1 is the nucleotide sequence of DENV-1 Hawaii C-prM-E.

SEQ ID NO: 2 is the nucleotide sequence of DENV-1 Hawaii prM-E.

SEQ ID NO: 3 is the nucleotide sequence of DENV-1 Hawaii mature capsid.

SEQ ID NO: 4 is the nucleotide sequence of DENV-1 Hawaii envelope.

SEQ ID NO: 5 is the nucleotide sequence of DENV-2 strain 16681 C-prM-E.

SEQ ID NO: 6 is the nucleotide sequence of DENV-3 strain H87C-prM-E.

SEQ ID NO: 7 is the nucleotide sequence of DENV-4 strain H241 C-prM-E.

SEQ ID NO: 8 is the nucleotide sequence of the FMDV 21A autocatalytic site.

SEQ ID NO: 9 is the nucleotide sequence of WNV NY99 C-prM-E. SEQ ID NO: 10 is the nucleotide sequence of WNV NY99 prM-E.

SEQ ID NO: 11 is the nucleotide sequence of WNV NY99 mature capsid.

### DETAILED DESCRIPTION

### I. Abbreviations

C flavivirus capsid protein

CFC cytokine flow cytometry

CNS central nervous system

DENV dengue virus

E flavivirus envelope protein

EΔTM flavivirus E protein lacking the transmembrane domain

EM electron microscopy

ffu focus forming units

FMDV foot and mouth disease virus

IFN interferon

IL interleukin

IS immune serum

JEV Japanese encephalitis virus

M flavivirus membrane protein

prM flavivirus premembrane protein

RT-PCR reverse transcriptase polymerase chain reaction

UTR untranslated region

VLP virus-like particle

WNV West Nile virus

YFV yellow fever virus

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Adjuvant: A substance or vehicle that non-specifically enhances the immune response to an antigen. Adjuvants can include a suspension of minerals (alum, aluminum hydroxide, or phosphate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. Exemplary biological adjuvants include IL-2, RANTES, GM-CSF, TNF-α, IFN-γ, G-CSF, LFA-3, CD72, B7-1, B7-2, OX-40L and 41 BBL.

Administer: As used herein, administering a composition (e.g. a VLP) to a subject means to give, apply or bring the composition into contact with the subject.

Administration can be accomplished by any of a number of routes, such as, for example, topical, oral, subcutaneous, intramuscular, intraperitoneal, intravenous, intrathecal and intramuscular.

Antigen: A compound, composition, or substance that can stimulate the production of antibodies or a T-cell response in an animal, including compositions that are injected or absorbed into an animal. An antigen reacts with the products of specific humoral or cellular immunity, including those induced by heterologous immunogens.

Capsid protein (C protein): One of three flavivirus structural proteins that forms the flavivirus particle. The C protein is a dimeric, alpha-helical protein with an unstructured N-terminus. In flavivirus particles, the C protein is found internal to the lipid bilayer and directly contacts the flavivirus genomic RNA.

Codon-optimized: A “codon-optimized” nucleic acid refers to a nucleic acid sequence that has been altered such that the codons are optimal for expression in a particular system (such as a particular species of group of species). For example, a nucleic acid sequence can be optimized for expression in yeast. Codon optimization does not alter the amino acid sequence of the encoded protein.

Conservative substitution: A substitution of one amino acid residue in a protein sequence for a different amino acid residue having similar biochemical properties. Typically, conservative substitutions have little to no impact on the activity of a resulting polypeptide. For example, ideally, a flavivirus protein including one or more conservative substitutions (for example no more than 2, 5, 10, 20, 30, 40, or 50 substitutions) retains the structure and function of the wild-type protein. A polypeptide can be produced to contain one or more conservative substitutions by manipulating the nucleotide sequence that encodes that polypeptide using, for example, standard procedures such as site-directed mutagenesis or PCR. In one example, such variants can be readily selected by testing antibody cross-reactivity or its ability to induce an immune response.

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

Envelope glycoprotein (E protein): A flavivirus structural protein that mediates binding of flavivirus virions to cellular receptors on host cells. The flavivirus E protein is required for membrane fusion, and is the primary antigen inducing protective immunity to flavivirus infection. Flavivirus E protein affects host range, tissue tropism and viral virulence. The flavivirus E protein contains three structural and functional domains, DI-DIII. In mature virus particles the E protein forms head to tail homodimers lying flat and forming a dense lattice on the viral surface.

Flavivirus structural protein: The capsid (C), premembrane (prM), and envelope (E) proteins of a flavivirus are the viral structural proteins. Flavivirus genomes consist of positive-sense RNAs that are roughly 11 kb in length. The genome has a 5′ cap, but lacks a 3′ polyadenylated tail (Wengler et al., Virology 89:423-437, 1978) and is translated into one polyprotein. The structural proteins (C, prM, and E) are at the amino-terminal end of the polyprotein followed by the non-structural proteins (NS1-5). The polyprotein is cleaved by virus and host derived proteases into individual proteins. The C protein forms the viral capsid while the prM and E proteins are embedded in the surrounding envelope (Russell et al., The Togaviruses: Biology, Structure, and Replication, Schlesinger, ed., Academic Press, 1980). The E protein functions in binding to host cell receptors resulting in receptor-mediated endocytosis. In the low pH of the endosome, the E protein undergoes a conformational change causing fusion between the viral envelope and the endosomal membranes. The prM protein is believed to stabilize the E protein until the virus exits the infected cell, at which time prM is cleaved to the mature M protein (Reviewed in Lindenbach and Rice, In: Fields Virology, Knipe and Howley, eds., Lippincott, Williams, and Wilkins, 991-1041, 2001).

FMDV 2A: The foot and mouth disease virus 2A autocatalytic site. In the context of the present disclosure, “2A” refers to the FMDV 2A autocatalytic site. In some embodiments, the FMDV 2A sequence is at least 95% identical to the nucleotide sequence of SEQ ID NO: 8. In particular examples, the 2A sequence comprises or consists of the nucleotide sequence of SEQ ID NO: 8 (agtaacttcgacctcctcaagttggcggga gacgttgagtccaaccccggacccgcc).

Immune response: A response of a cell of the immune system, such as a B-cell, T-cell, macrophage or polymorphonucleocyte, to a stimulus such as an antigen. An immune response can include any cell of the body involved in a host defense response for example, an epithelial cell that secretes an interferon or a cytokine. An immune response includes, but is not limited to, an innate immune response or inflammation.

Immunize: To render a subject protected from an infectious disease, such as by vaccination.

Immunomodulator: An agent that alters (such as suppresses or enhances) immune responses. In the context of the present disclosure, the immunomodulator is an immune response protein, or fragment thereof. In particular examples, the immune response protein is interleukin (IL)-1, IL-12, interferon (IFN)-α, IFN-β or IFN-γ, or a fragment thereof. In some embodiments of the present disclosure, an immunomodulator is fused with the coding region of C-prM-E, such as at the 3′ end of the E coding region.

Isolated: An “isolated” or “purified” biological component (such as a nucleic acid, peptide, protein, protein complex, or virus-like particle) has been substantially separated, produced apart from, or purified away from other biological components in the cell of the organism in which the component naturally occurs, that is, other chromosomal and extrachromosomal DNA and RNA, and proteins. Nucleic acids, peptides and proteins that have been “isolated” or “purified” thus include nucleic acids and proteins purified by standard purification methods. The term also embraces nucleic acids, peptides and proteins prepared by recombinant expression in a host cell, as well as chemically synthesized nucleic acids or proteins. The term “isolated” or “purified” does not require absolute purity; rather, it is intended as a relative term. Thus, for example, an isolated biological component is one in which the biological component is more enriched than the biological component is in its natural environment within a cell, or other production vessel. Preferably, a preparation is purified such that the biological component represents at least 50%, such as at least 70%, at least 90%, at least 95%, or greater, of the total biological component content of the preparation.

Mechanical lysis: Refers to any mechanical means for cell disruption, leading to release of biological components from a cell (such as release of proteins from a yeast cell). In one non-limiting embodiment, mechanical lysis is carried out using Emulsiflex™ C-3. In some examples, mechanical lysis is carried out with an average pressure of about 20,000 to about 25, 000 psi.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compositions, such as one or more flavivirus vaccines, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Plasmid: A circular nucleic acid molecule capable of autonomous replication in a host cell.

Premembrane protein (prM protein): A flavivirus structural protein. The prM protein is an approximately 25 kDa protein that is the intracellular precursor for the membrane (M) protein. prM is believed to stabilize the E protein during transport of the immature virion to the cell surface. When the virus exits the infected cell, the prM protein is cleaved to the mature M protein, which is part of the viral envelope (Reviewed in Lindenbach and Rice, In: Fields Virology, Knipe and Howley, eds., Lippincott, Williams, and Wilkins, 991-1041, 2001).

Promoter: A promoter is an array of nucleic acid control sequences which direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor). In some embodiments herein, the promoter is suitable for expression in yeast cells. In particular non-limiting examples, the promoter is the AOX promoter or the GAPDH promoter.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Natl. Acad. Sci., 85:2444, 1988); Higgins and Sharp (Gene, 73:237-44, 1988); Higgins and Sharp (CABIOS, 5:151-53, 1989); Corpet et al. (Nuc. Acids Res., 16:10881-90, 1988); Huang et al. (Comp. Appls. Biosci., 8:155-65, 1992); and Pearson et al. (Meth. Mol. Biol., 24:307-31, 1994). Altschul et al. (Nature Genet., 6:119-29, 1994) presents a detailed consideration of sequence alignment methods and homology calculations.

The alignment tools ALIGN (Myers and Miller, CABIOS 4:11-17, 1989) or LFASTA (Pearson and Lipman, Proc. Natl. Acad. Sci. 85:2444-2448, 1988) may be used to perform sequence comparisons (Internet Program© 1996, W. R. Pearson and the University of Virginia, “fasta20u63” version 2.0u63, release date December 1996). ALIGN compares entire sequences against one another, while LFASTA compares regions of local similarity. These alignment tools and their respective tutorials are available on the Internet at the NCSA website. Alternatively, for comparisons of amino acid sequences of greater than about 30 amino acids, the “Blast 2 sequences” function can be employed using the default BLOSUM62 matrix set to default parameters, (gap existence cost of 11, and a per residue gap cost of 1). When aligning short peptides (fewer than around 30 amino acids), the alignment should be performed using the “Blast 2 sequences” function, employing the PAM30 matrix set to default parameters (open gap 9, extension gap 1 penalties). The BLAST sequence comparison system is available, for instance, from the NCBI web site; see also Altschul et al., J. Mol. Biol., 215:403-10, 1990; Gish and States, Nature Genet., 3:266-72, 1993; Madden et al., Meth. Enzymol., 266:131-41, 1996; Altschul et al., Nucleic Acids Res., 25:3389-402, 1997; and Zhang and Madden, Genome Res., 7:649-56, 1997.

Orthologs (equivalent to proteins of other species) of proteins are in some instances characterized by possession of greater than 75% sequence identity counted over the full-length alignment with the amino acid sequence of specific protein using ALIGN set to default parameters. Proteins with even greater similarity to a reference sequence will show increasing percentage identities when assessed by this method, such as at least 80%, at least 85%, at least 90%, at least 92%, at least 95%, at least 98%, or at least 99% sequence identity. In addition, sequence identity can be compared over the full length of one or both binding domains of the disclosed fusion proteins.

When significantly less than the entire sequence is being compared for sequence identity, homologous sequences will typically possess at least 80% sequence identity over short windows of 10-20, and may possess sequence identities of at least 85%, at least 90%, at least 95%, 96%, 97%, 98%, or at least 99% depending on their similarity to the reference sequence. Sequence identity over such short windows can be determined using LFASTA; methods are described at the NCBI website. One of skill in the art will appreciate that these sequence identity ranges are provided for guidance only; it is entirely possible that strongly significant homologs could be obtained that fall outside of the ranges provided. Similar homology concepts apply for nucleic acids as are described for protein. An alternative indication that two nucleic acid molecules are closely related is that the two molecules hybridize to each other under stringent conditions.

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

Subject: Living multi-cellular vertebrate organisms, a category that includes both human and non-human mammals (such as mice, rats, rabbits, sheep, horses, cows, and non-human primates).

Therapeutically effective amount: A quantity of a specified agent (such as a flavivirus VLP) sufficient to achieve a desired effect in a subject being treated with that agent. For example, this may be the amount of a flavivirus vaccine useful for eliciting an immune response in a subject and/or for preventing infection by a flavivirus. Ideally, in the context of the present disclosure, a therapeutically effective amount of a flavivirus vaccine is an amount sufficient to increase resistance to, prevent, ameliorate, and/or treat infection caused by a flavivirus in a subject without causing a substantial cytotoxic effect in the subject. The effective amount of a flavivirus vaccine useful for increasing resistance to, preventing, ameliorating, and/or treating infection in a subject will be dependent on, for example, the subject being treated, the manner of administration of the therapeutic composition and other factors.

Transformed: A “transformed” cell is a cell (such as a yeast cell, for example a Pichia pastoris cell) into which has been introduced a nucleic acid molecule by molecular biology techniques. The term encompasses all techniques by which a nucleic acid molecule might be introduced into such a cell, including transfection with viral vectors, transformation with plasmid vectors, and introduction of naked DNA by electroporation, lipofection, and particle gun acceleration.

Vaccine: A preparation of immunogenic material capable of stimulating an immune response, administered for the prevention, amelioration, or treatment of infectious or other types of disease. The immunogenic material may include attenuated or killed microorganisms (such as bacteria or viruses), or antigenic proteins (including VLPs), peptides or DNA derived from them. An attenuated vaccine is a virulent organism that has been modified to produce a less virulent form, but nevertheless retains the ability to elicit antibodies and cell-mediated immunity against the virulent form. A killed vaccine is a previously virulent microorganism that has been killed with chemicals or heat, but elicits antibodies against the virulent microorganism. Vaccines may elicit both prophylactic (preventative) and therapeutic responses. Methods of administration vary according to the vaccine, but may include inoculation, ingestion, inhalation or other forms of administration. Vaccines may be administered with an adjuvant to boost the immune response.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes.

Virus-like particle (VLP): Virus particles made up of one of more viral structural proteins, but lacking the viral genome. Because VLPs lack a viral genome, they are non-infectious. In addition, VLPs can often be produced by heterologous expression and can be easily purified. In some embodiments disclosed herein, flavivirus VLPs include the three flavivirus structural proteins (C, prM and E).

Yeast: Single-celled fungi that reproduce asexually. In the context of the present disclosure, any appropriate type of yeast species or strain can be used to express flavivirus VLPs. In some embodiments, the yeast is Pichia pastoris, Saccharomyces cerevisiae or Schizosaccharomyces pombe. In particular examples, the yeast is the X33, GlycoSwitch™, Pichia Expression or GS115 strain of Pichia pastoris.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview of Several Embodiments

Disclosed herein is a method for the production of flavivirus VLPs in a yeast system. In some embodiments, flavivirus structural proteins are expressed in yeast, such as in a Pichia pastoris strain, and isolated using pressurized mechanical lysis. Also disclosed herein are nucleic acid molecules encoding flavivirus VLPs comprising the C protein, the prM protein and at least one E protein (or an E protein lacking the transmembrane domain). In some embodiments, the nucleic acid molecules further include at least one (for example, 2-4) FMDV 2A autocatalytic site. When present, the FMDV 2A autocatalytic site or sites can be between any two flavivirus proteins, such as between C and prM, between prM and E, and/or between two E proteins. The disclosed VLPs can be used as immunogenic compositions for the prevention or treatment of flavivirus infection.

Provided herein are isolated nucleic acid molecules encoding a flavivirus capsid (C) protein; a flavivirus premembrane (prM) protein; and at least one flavivirus envelope (E) protein or a flavivirus E protein lacking the transmembrane domain (EΔTM), wherein the nucleic acid molecule further encodes at least one foot and mouth disease virus (FMDV) 2A autocatalytic site (2A) positioned between two flavivirus protein coding sequences. Expression of the nucleic acid molecules in a host cell produces flavivirus VLPs.

In some embodiments, the nucleic acid molecule has the following formula: C-2A-prM-E; C-prM-2A-E; C-2A-prM-2A-E; C-2A-prM-E₁-E₂-E₃-E₄; C-2A-prM-2A-E₁-E₂-E₃-E₄; C-prM-E₁-2A-E₂-2A-E₃-2A-E₄; C-2A-prM-E₁-2A-E₂-2A-E₃-2A-E₄; C-prM-2A-E₁-2A-E₂-2A-E₃-2A-E₄; C-2A-prM-2A-E₁-2A-E₂-2A-E₃-2A-E₄; C-2A-prM-EΔTM; C-prM-2A-EΔTM; or C-2A-prM-2A-EΔTM.

In other embodiments, the nucleic acid molecule has the following formula: C-2A-prM-E₁ΔTM-E₂ΔTM-E₃ΔTM-E₄ΔTM; C-2A-prM-2A-E₁ΔTM-E₂ΔTM-E₃ΔTM-E₄ΔTM; C-prM-E₁ΔTM-2A-E₂ΔTM-2A-E₃ΔTM-2A-E₄ΔTM; C-2A-prM-E₁ΔTM-2A-E₂ΔTM-2A-E₃ΔTM-2A-E₄ΔT; C-prM-2A-E₁ΔTM-2A-E₂ΔTM-2A-E₃ΔTM-2A-E₄ΔT; or C-2A-prM-2A-E₁ΔTM-2A-E₂ΔTM-2A-E₃ΔTM-2A-E₄ΔTM. In any of the above examples, EₙΔTM can be substituted for Eₙ (where n=1, 2, 3 or 4).

E₁, E₂, E₃ and E₄ or E₁ΔTM, E₂ΔTM, E₃ΔTM and E₄ΔTM can each be from a different flavivirus or can represent multiple copies of an E protein from one or more flaviviruses.

In some embodiments, the nucleic acid molecule comprises a nucleotide sequence at least 85%, at least 90%, at least 95%, at least 96%, at least 97%, at least 98% or at least 99% identical to any one of SEQ ID NOs: 1-7 and 9-11. In particular examples, the nucleotide sequence of the nucleic acid molecule comprises any one of SEQ ID NOs: 1-7 and 9-11.

In some examples, the FMDV 21A autocatalytic site is encoded by the nucleotide sequence of SEQ ID NO: 8.

In some embodiments, the flavivirus is selected from dengue virus type 1, dengue virus type 2, dengue virus type 3, dengue virus type 4, West Nile virus, Japanese encephalitis virus, yellow fever virus and St. Louis encephalitis virus.

In some examples, E₁, E₂, E₃ and E₄, or E₁ΔTM, E₂ΔTM, E₃ΔTM and E₄ΔTM, are each from a different flavivirus. In other examples, E₁, E₂, E₃ and E₄, or E₁ΔTM, E₂ΔTM, E₃ΔTM and E₄ΔTM, represent four copies of the E protein from a single flavivirus. Alternatively, E₁, E₂, E₃ and E₄, or E₁ΔTM, E₂ATM, E₃ΔTM and E₄ΔTM, represent multiple copies of an E protein from one or more flaviviruses (such as two copies from two different flaviviruses, or three copies from a first flavivirus a single copy from a second flavivirus). In one non-limiting example, E₁, E₂, E₃ and E₄, or E₁ΔTM, E₂ΔTM, E₃ΔTM and E₄ΔTM, are respectively from dengue virus type 1, dengue virus type 2, dengue virus type 3 and dengue virus type 4. In any of the above examples, EₙΔTM can be substituted for Eₙ (where n=1, 2, 3 or 4).

In some embodiments, the nucleic acid molecule further encodes an immunomodulator. In some examples, the immunomodulator comprises interleukin (IL)-1, IL-12, interferon (IFN)-α, IFN-β or IFN-γ or a fragment thereof.

In some embodiments, the nucleic acid molecule further encodes a tag, such as a tag to facilitate detection or isolation of the encoded protein. In particular examples, the tag is a His tag, a myc tag, a FLAG tag, an HA tag or a fluorescent protein (e.g., green fluorescent protein or a derivative thereof).

In some embodiments, the nucleotide sequence of the nucleic acid molecule is codon-optimized for expression in yeast.

VLPs encoded by the disclosed nucleic acid molecules are also provided by the present disclosure.

Also provided herein are vectors comprising the disclosed nucleic acid molecules. In some embodiments, the vector further includes a promoter operably linked to the nucleic acid molecule encoding the flavivirus C, prM and E proteins. In some examples, the promoter is the phosphoglycerate kinase (PGK) promoter, glyceraldehyde-3-phosphate dehydrogenase (GAPD) promoter, GAL1-10 promoter, methanol oxidase promoter, ADH1 promoter, CYC1 promoter, TDH3 promoter, or TEF promoter. In some embodiments, the vector further includes a selectable marker, a transcription termination sequence, or both.

Further provided are host cells containing the vectors disclosed herein. In some embodiments, the host cell is a yeast cell. In some examples, the yeast is Pichia pastoris, Saccharomyces cerevisiae or Schizosaccharomyces pombe. In specific non-limiting examples, the yeast is the X33, GlycoSwitch™, Pichia Expression or GS115 strain of Pichia pastoris. The Pichia Expression and GlycoSwitch™ strains are commercially available from Research Corporation Technologies (Tucson, Ariz.). Pichia Expression expresses proteins without glycosylation or low level yeast glycosylation. The GlycoSwitch™ strain produces proteins with human-like glycosylation.

Also provided is a method of producing VLPs by transforming yeast cells with a nucleic acid molecule or vector disclosed herein; cultivating the transformed yeast cells under conditions sufficient to allow for expression of the flavivirus proteins and formation of VLPs; subjecting the yeast cells to pressurized mechanical lysis; and isolating the VLPs from the yeast cell lysis.

Further provided is a method of producing flavivirus VLPs by transforming yeast cells with a nucleic acid molecule encoding a flavivirus premembrane (prM) protein; and at least one flavivirus envelope (E) protein or a flavivirus E protein lacking the transmembrane domain (EΔTM); cultivating the transformed yeast cells under conditions sufficient to allow for expression of the flavivirus proteins and formation of VLPs; subjecting the yeast cells to pressurized mechanical lysis; and isolating the VLPs from the yeast cell lysis.

In some examples, pressurized mechanical lysis is carried out with an average pressure of about 20,000 to about 25, 000 psi, such as about 20,000 psi, about 21,000 psi, about 22,000 psi, about 23,000 psi, about 24,000 psi or about 25,000 psi. Mechanical lysis can be performed using, for example, Emulsiflex™ C-3.

In some embodiments of the method, the nucleic acid molecule further encodes a flavivirus capsid (C) protein, or at least one FMDV 2A autocatalytic site (2A) positioned between two flavivirus protein coding sequences, or both. In particular examples of the method, the nucleic acid molecule has the following formula: C-2A-prM-E; C-prM-2A-E; C-2A-prM-2A-E; C-2A-prM-E₁-E₂-E₃-E₄; C-2A-prM-2A-E₁-E₂-E₃-E₄; C-prM-E₁-2A-E₂-2A-E₃-2A-E₄; C-2A-prM-E₁-2A-E₂-2A-E₃-2A-E₄; C-prM-2A-E₁-2A-E₂-2A-E₃-2A-E₄; C-2A-prM-2A-E₁-2A-E₂-2A-E₃-2A-E₄; C-2A-prM-EΔTM; C-prM-2A-EΔTM; or C-2A-prM-2A-EΔTM.

In other examples, the nucleic acid molecule has the following formula: C-2A-prM-E₁ΔTM-E₂ΔTM-E₃ΔTM-E₄ΔTM; C-2A-prM-2A-E₁ΔTM-E₂ΔTM-E₃ΔTM-E₄ΔTM; C-prM-E₁ΔTM-2A-E₂ΔTM-2A-E₃ΔTM-2A-E₄ΔTM; C-2A-prM-E₁ΔTM-2A-E₂ΔTM-2A-E₃ΔTM-2A-E₄ΔT; C-prM-2A-E₁ΔTM-2A-E₂ΔTM-2A-E₃ΔTM-2A-E₄ΔT; or C-2A-prM-2A-E₁ΔTM-2A-E₂ΔTM-2A-E₃ΔTM-2A-E₄ΔTM. In any of the above examples, EₙΔTM can be substituted for Eₙ (where n=1, 2, 3 or 4).

E₁, E₂, E₃ and E₄, or E₁ΔTM, E₂ΔTM, E₃ΔTM and E₄ΔTM, can each be from a different flavivirus or can represent multiple copies of an E protein from one or more flaviviruses.

In some embodiments of the method, the flavivirus is selected from dengue virus type 1, dengue virus type 2, dengue virus type 3, dengue virus type 4, West Nile virus, Japanese encephalitis virus, yellow fever virus and St. Louis encephalitis virus.

In some examples, E₁, E₂, E₃ and E₄, or E₁ΔTM, E₂ΔTM, E₃ΔTM and E₄ΔTM, are each from a different flavivirus. In other examples, E₁, E₂, E₃ and E₄, or E₁ΔTM, E₂ΔTM, E₃ΔTM and E₄ΔTM, represent four copies of the E protein from a single flavivirus. Alternatively, E₁, E₂, E₃ and E₄, or E₁ΔTM, E₂ΔTM, E₃ΔTM and E₄ΔTM, represent multiple copies of an E protein from one or more flaviviruses (such as two copies from two different flaviviruses, or three copies from a first flavivirus a single copy from a second flavivirus). In one non-limiting example, E₁, E₂, E₃ and E₄, or E₁ΔTM, E₂ΔTM, E₃ΔTM and E₄ΔTM, are respectively from dengue virus type 1, dengue virus type 2, dengue virus type 3 and dengue virus type 4. In any of the above examples, EₙΔTM can be substituted for Eₙ (where n=1, 2, 3 or 4).

In some embodiments of the methods, the nucleic acid molecule comprises a nucleotide sequence at least 85%, at least 90%, at least 95%, at least 96%, at least 97%, at least 98% or at least 99% identical to any one of SEQ ID NOs: 1-7 and 9-11. In particular embodiments, the nucleic acid molecule comprises SEQ ID NO: 1 (DENV-1 Hawaii C-prM-E), SEQ ID NO: 2 (DENV-1 Hawaii prM-E), SEQ ID NO: 3 (DENV-1 Hawaii C), SEQ ID NO: 4 (DENV-1 Hawaii E), SEQ ID NO: 5 (DENV-2 strain 16681 C-prM-E), SEQ ID NO: 6 (DENV-3 strain H87 C-prM-E), SEQ ID NO: 7 (DENV-4 strain H241 C-prM-E), SEQ ID NO: 9 (WNV NY99 C-prM-E), SEQ ID NO: 10 (WNV NY99 prM-E) or SEQ ID NO: 11 (WNV NY99 C), or any combination thereof.

In some examples, the FMDV 21A autocatalytic site is encoded by the nucleotide sequence of SEQ ID NO: 8.

In some embodiments of the method, the nucleic acid molecule further encodes an immunomodulator. In some examples, the immunomodulator comprises interleukin (IL)-1, IL-12, interferon (IFN)-α, IFN-β or IFN-γ or a fragment thereof.

In some embodiments of the method, the nucleic acid molecule further encodes a tag, such as a tag to facilitate detection or isolation of the encoded protein. In particular examples, the tag is a His tag, a myc tag, a FLAG tag, an HA tag or a fluorescent protein (e.g., green fluorescent protein or a derivative thereof).

In some embodiments of the method, the nucleotide sequence of the nucleic acid molecule is codon-optimized for expression in yeast.

In some embodiments of the method, the yeast is Pichia pastoris, Saccharomyces cerevisiae or Schizosaccharomyces pombe. In particular examples, the yeast is the X33, GlycoSwitch™, Pichia Expression or GS115 strain of Pichia pastoris.

In some embodiments of the method, isolating the VLPs from the yeast cell lysis comprises sucrose-gradient centrifugation.

Also provided are isolated flavivirus VLPs produced by the method disclosed herein.

Further provided are compositions comprising the disclosed VLPs and a pharmaceutically acceptable carrier. In some embodiments, the compositions further include an adjuvant.

Methods of eliciting an immune response in a subject against flavivirus are also provided by the present disclosure. In some embodiments, the method includes administering to the subject a VLP (or composition thereof) as disclosed herein.

### IV. Flaviviruses and Virus-Like Particles (VLPs)

The family Flaviviridae consists of positive-strand RNA viruses with members including, but not limited to, human pathogens dengue virus (DENV), West Nile virus (WNV), yellow fever virus (YFV), and Japanese encephalitis virus (JEV). The virion consists of an envelope surrounding an icosahedral capsid of approximately 50 nm. The genome is a single, capped, positive-sense RNA molecule of approximately 11 kb. The genomic RNA encodes a single open reading frame that is flanked by 5′ and 3′ untranslated regions (UTR) containing replication promoters and translation signals. The polyprotein is processed into ten polypeptides by cellular and viral proteases. Three of these proteins are the structural components required for capsid formation (capsid protein—C) and assembly into viral particles (premembrane prM) and envelope proteins—E).

Virus-like particles (VLPs), noninfectious virions composed of structural proteins but lacking a viral genome, have been produced in various cell expression systems. A major advantage of a VLP-based vaccine compared to live-attenuated virus is that a VLP expresses similar antigenic conformations without potential adverse effects of a live-attenuated virus, such as reversion. Further, VLPs can be used in immunocompromised populations, whereas inactivated and live-attenuated virus vaccines pose significant hazards in these patients. VLPs have the potential for activating both the endogenous and exogenous antigen pathways leading to the presentation of viral peptides by major histocompatibility complex (MHC) class I and class II molecules. VLPs bind and enter cells using appropriate surface receptors. Following cell entry, VLP proteins are processed and presented on MHC class I molecules, promoting presentation to T-cells by antigen-presenting cells. In addition, cell-free VLPs bound with antibodies can be taken up by phagocytic cells via Fc receptors, thus increasing MHC class II presentation.

Appropriate presentation of viral antigens leads to more efficient antibody production. Antigens expressed in their native conformational form can elicit more effective antibody responses than proteins in their non-native forms. Many neutralizing antibodies directed against viruses are elicited against conformational epitopes present only in the native form of envelopes, and some are exposed only after binding to receptors during entry. For example, recombinant protein vaccines against flavivirus envelope glycoproteins elicit high titer antibodies. However, these antibodies often only neutralize the homologous flavivirus and not other viral isolates. Particle-based vaccines, containing native forms of envelope (E), in addition to other viral antigens, have the potential to induce strong humoral and cell-mediated responses to multiple viral proteins.

To date, most flavivirus VLPs have consisted of two structural proteins (prM and E) produced from mammalian cells. Limitations of this approach include low levels and variability of protein expression; difficult production for scale-up; and very high cost. To circumvent these difficulties, VLPs were expressed in yeast. The benefits of using a yeast-based expression system are many fold, including high levels of expression, controlled induction of protein expression, easy scale-up, and very low cost. In some embodiments disclosed herein, the flavivirus VLPs include the C protein, which will present more antigenic targets as well as a more complete virion structure.

In summary, VLP vaccines are advantageous over traditional vaccines for at least the following reasons:

(1) shorter lead times for development of “new-to-the-world” vaccines—recombinant DNA technology facilitates rapid serotype matching and shorter lead times;

(2) rapid response—yeast-based methods using large fermenters provide for rapid response (scalable and transferable) surge capacity;

(3) stability—expression cassettes stably integrate into the yeast genome, allowing repeated expression;

(4) high immunogenicity—VLP vaccines provide the correct three-dimensional antigenic conformation of the viral proteins (e.g. C, prM/M and E) and “native-like” presentation of antigens to the immune system; and

(5) safety—VLP vaccines lack infectious genetic material, thus there can be no replication, reversion of attenuation, or shedding of infectious virus.

### V. VLP Compositions and Administration Thereof

Flavivirus VLPs, or compositions thereof, can be administered to a subject by any of the routes normally used for introducing recombinant virus into a subject. Methods of administration include, but are not limited to, intradermal, intramuscular, intraperitoneal, parenteral, intravenous, subcutaneous, vaginal, rectal, intranasal, inhalation or oral. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions. Injection solutions and suspensions can be prepared from sterile powders, granules, and tablets of the kind previously described. Administration can be systemic or local.

Flavivirus VLPs, or compositions thereof, are administered in any suitable manner, such as with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Administration can be accomplished by single or multiple doses. The dose administered to a subject in the context of the present disclosure should be sufficient to induce a beneficial therapeutic response in a subject over time, or to inhibit or prevent flavivirus infection. The dose required will vary from subject to subject depending on the species, age, weight and general condition of the subject, the severity of the infection being treated, the particular composition being used and its mode of administration. An appropriate dose can be determined by one of ordinary skill in the art using only routine experimentation.

In some embodiments, the dose of dengue VLP is about 1 to about 100 μg. In particular examples, the dose of dengue VLP is about 5, about 10, about 15, about 20, about 25, or about 50 μg.

Provided herein are pharmaceutical compositions which include a therapeutically effective amount of the flavivirus VLPs alone or in combination with a pharmaceutically acceptable carrier. Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, and magnesium carbonate. Any of the common pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used with the compositions and methods provided herein are normal saline and sesame oil.

The flavivirus VLPs described herein can be administered alone or in combination with other therapeutic agents to enhance antigenicity. For example, the flavivirus VLPs can be administered with an adjuvant, such as Freund incomplete adjuvant or Freund's complete adjuvant.

Optionally, one or more cytokines, such as IL-2, IL-6, IL-12, RANTES, GM-CSF, TNF-α, or IFN-γ, one or more growth factors, such as GM-CSF or G-CSF; one or more molecules such as OX-40L or 41 BBL, or combinations of these molecules, can be used as biological adjuvants (see, for example, Salgaller et al., 1998, J. Surg. Oncol. 68(2):122-38; Lotze et al., 2000, Cancer J. Sci. Am. 6(Suppl 1):S61-6; Cao et al., 1998, Stem Cells 16(Suppl 1):251-60; Kuiper et al., 2000, Adv. Exp. Med. Biol. 465:381-90). These molecules can be administered systemically (or locally) to the host.

### VI. Production of VLPs in Yeast

Disclosed herein is a yeast-based system for expression of flavivirus VLPs. In some embodiments, the method includes transforming a yeast cell with a nucleic acid molecule encoding flavivirus structural proteins; cultivating the transformed yeast cells under conditions sufficient to allow for expression of the flavivirus proteins and formation of VLPs; subjecting the yeast cells to pressurized mechanical lysis; and isolating the VLPs from the yeast cell lysis. In some embodiments, the flavivirus structural proteins include the C, prM and E proteins. Alternatively, the flavivirus structural proteins include only the prM and E proteins. In some cases, the E protein lacks the transmembrane domain (EΔTM).

The use of Pichia pastoris for expression of flavivirus VLPs is exemplified herein, however, any suitable yeast can be used, such as Saccharomyces cerevisiae or Schizosaccharomyces pombe.

In one non-limiting embodiment, Pichia pastoris is transformed with a vector (such as the pPIC3.5 vector) encoding flavivirus C-prM-E. Individual colonies with integrated vector are grown in YEPD broth for approximately 8 hours at 30° C. The yeast culture is diluted in minimal growth media (MGY: 1.3% YNB, 1.2% glycerol, 4×10⁻⁵% biotin) and grown for about 16 hours. Protein expression is induced by dilution of the MGY culture into minimal methanol media (MM: 1.3% YNB, 4×10⁻⁵% biotin, 0.75% methanol). After approximately 24 hours, yeast cells are mechanically lysed using Emulsiflex™ C-3. In one non-limiting embodiment, the lysis conditions are as follows:

The Pichia pastoris culture is centrifuged to isolate cells. The pellet is thoroughly resuspended in ice-cold 10-25 mL PBS+1% BSA. The Emulsiflex™ C-3 lysis machine is primed with 50 mL PBS+1% BSA and the resuspended yeast cell mixture is run through the machine 7-10 times with average pressure of 20,000-25,000 psi. If necessary, lysis can be verified by light microscopy at 20× magnification. An aliquot of the lysate is removed and stored at −80° C. The remaining lysate is centrifuged at 10,000×g. The supernatant, referred to as S10-VLP, is removed to another tube. The pellet is resuspended in PBS+1% BSA and stored at −80° C. An aliquot of S10-VLP is removed and stored at −80° C. The remaining S10-VLP can be purified by sucrose gradient centrifugation (as described in Example 3).

Yeast lysates are cleared of debris, then VLPs are isolated by sucrose gradient centrifugation. Production of VLP proteins is verified, such as by Western blot.

In some examples, following mechanical lysis, lysates are clarified by low-speed centrifugation (8-10,000×g). Lysates are then layered on a 5-50% sucrose gradient and centrifuged at high speed to sediment VLPs. Size exclusion spin columns can also be used. Cell lysates and purified VLPs are separated by SDS-PAGE and proteins are transferred to PVDF membranes.

To determine the stoichiometry of the structural proteins in the VLPs. the quantity of each constituent protein per VLP can be compared to the amount in infectious virus. For example, VLPs can be isolated from yeast and infectious virus can be isolated from infected BHK cell culture media by purifying particles by centrifugation, as described above. Equivalent quantities of VLP and virus can be separated by SDS-PAGE, as determined by protein concentration, and proteins can be detected by Western blot. Densitometric analysis of the bands can be used to quantify the amount of C, prM, and E present in VLPs, which can be compared to infectious virus.

To determine if the VLPs produced by P. pastoris (or another type of yeast) physically resemble the infectious viruses from which they were derived, the VLPs can be visually inspected and compared by transmission electron microscopy.

Samples of isolated VLPs and infectious virus can be fixed with glutaraldehyde and stained with uranyl acetate for electron microscopy. The overall morphology can be compared, as well as the dimensions of the VLPs and infectious virus.

The following examples are provided to illustrate certain particular features and/or embodiments. These examples should not be construed to limit the disclosure to the particular features or embodiments described.

### EXAMPLES

### Example 1

### Expression and Characterization of WNV VLPs

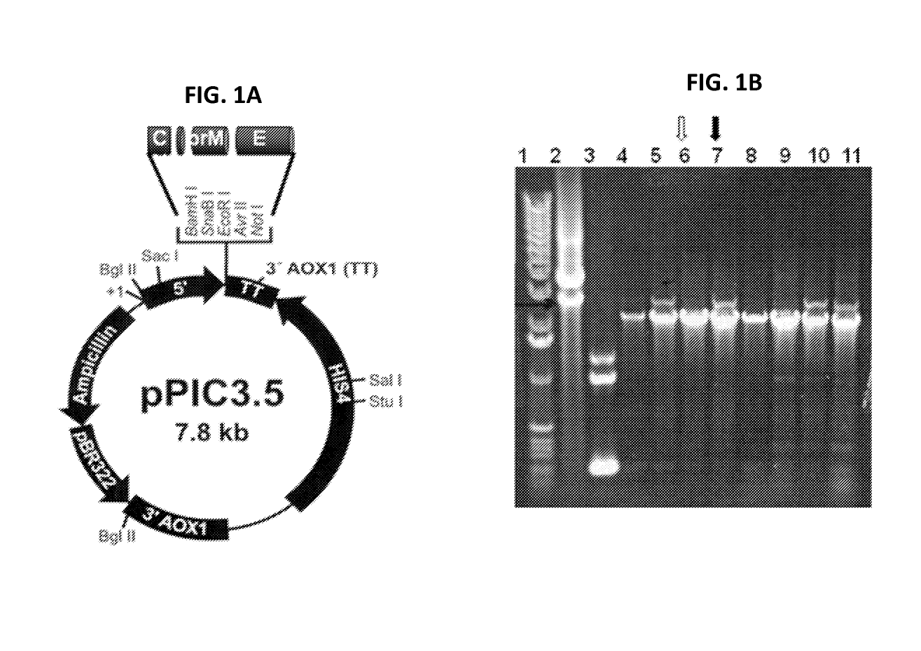

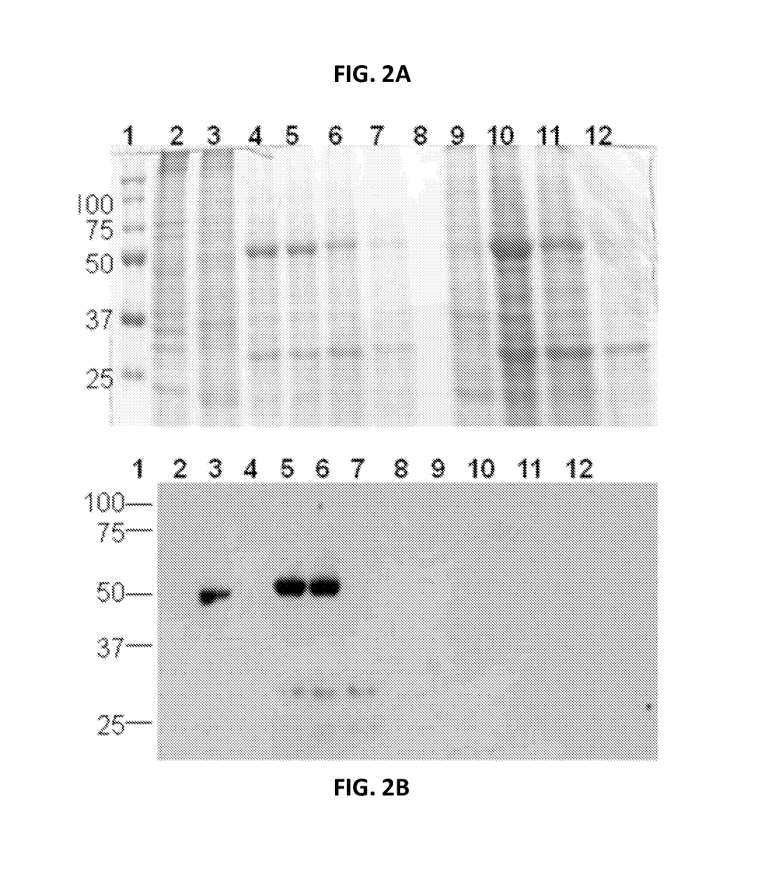

The C-prM-E coding region from WNV NY99 was cloned into P. pastoris vector pPIC3.5 (Invitrogen) and integration into P. pastoris was verified by PCR (FIG. 1). Expression of E protein was confirmed by Western blot (FIG. 2). Gradient centrifugation was performed, which revealed two differently sedimenting populations, one heavy and one light. These two populations are compared to infectious virions. Incorporation of C and prM proteins into VLPs is verified by centrifugation and Western blot. To validate the structure of the VLPs, electron microscopy (EM) is performed. In addition, immuno-EM is carried out using antibodies to C, prM and E to confirm their inclusion in the VLPs. Experiments are performed to determine if VLPs can be tagged for localization and infection studies, and to determine whether immunomodulators can be added to the VLPs to increase immunogenicity.

Constructs are tested in different yeast strains and with different expression protocols. Codon-optimized genetic sequences are also tested to enhance expression in yeast.

### Example 2

### Characterization of Immune Responses to WNV VLPs

### Dosing Study in BALB/c Mice

BALB/C mice will be vaccinated by the intraperitoneal route in groups with different amounts of WNV VLPs (0, 10 μg, and 100 μg). Table 1 describes the schedule of vaccinations and assays. Serum samples will be obtained every two weeks (0-16) to measure WNV-specific antibodies. At week 14, animals will be divided and half will be sacrificed for cellular analyses on lymph nodes and spleens. The second half will be monitored until week 32 to measure longevity of immune response. The most effective vaccines are those that elicit both arms of the immune system. VLPs elicit both humoral and cellular immunity in mice.

TABLE 1
Immunization Schedule and Sample Regimen

| Assay/Procedure | 0 | 2 | 4 | 6 | 8 | 10 | 12 | 14 | 16 | 24 | 32 |
|---|---|---|---|---|---|---|---|---|---|---|---|
| Vaccination | X | | X | | | | | | | | |
| ELISA | X | X | X | X | X | X | X | X | X | X | X |
| Ab maturation | X | | | X | X | X | X | X | X | X | X |
| SPR binding | | | | X | X | X | X | X | X | X | X |
| Neutralization | X | | | X | X | X | X | X | X | X | X |
| ADE | X | | | X | X | X | X | X | X | X | X |
| IFN-γ ELISPOT | X | | | | | X | | X | X | X | X |
| FACS | X | | | | | X | | X | X | X | X |
| Luminex ™ | X | | | | | X | | X | X | X | X |

### Measurement of Vaccine Elicited Humoral and Cellular Immunity

Antibody production will be analyzed by: 1) characterization of the quantitative and qualitative properties of WNV E-specific antibody responses; 2) characterization of kinetic binding properties of polyclonal antibodies; and 3) measurement of neutralizing antibodies.

### Measurement of Breadth, Specificity, and Maturation of T Cell Responses Elicited by WNV VLPs

Using overlapping 15-mer peptides, an IFN-γ ELISPOT will be used as a first screen. The second assay used will be polychromatic cytokine flow cytometry (CFC), which provides a more qualitative analysis of cellular responses. The studies will examine (1) the strength and breadth of responses, and (2) the maturation of T cell phenotypes following vaccination. Finally, cytokine activity in the spleens will be measured using a 32-plex Luminex™.

### Small Animal WNV Challenge

Using the information garnered through the dosing study described above, mice will be vaccinated with a dose of WNV VLP that induces neutralizing antibody titers. Two sets of vaccinations will be performed, with animals receiving a 2-dose regimen. Mice will be challenged with 1000 focus forming units (ffu) of WNV NY99 in a volume of 0.1 ml by the intraperitoneal route. WNV will be diluted in a filtered solution of 10% fetal bovine serum (FBS) in phosphate buffered saline (PBS) prior to the mice infections. Mice will be weighed daily to determine an individual animal's percent weight loss, and monitored to determine the severity of sickness, for which a devised a score method will be used. Moribund mice (severe lethargy, hunched posture, and ruffled fur) will be euthanized by CO₂ asphyxiation and recorded as dead for the next day. Serum samples will be obtained on days 2, 4, 6, 8, 12 and 16. The serum will be analyzed for the presence of virus by plaque focus assay, according to standard procedures.

### Pathology of Vaccinated-Challenged Animals

To fully evaluate the efficacy of the VLP vaccine, the effects of infectious virus challenge in mice will be examined. A series of pathology analyses will be conducted to determine if virus infection occurred, its location, and its potential damage. The immune response in specific organs will also be determined by staining and immunohistochemical analyses. These approaches will allow for a determination of the protective mechanism of the vaccine by differentiating between protection from initial virus infection or subsequent disease manifestations. Further, this approach will allow for a determination of the temporal and spatial distribution of infecting virus as well as infiltrating immune cells.

Role of Antibodies in Vaccine Protection from Infectious Virus Challenge

To investigate the mechanism for protection, mice will be vaccinated and passive transfer experiments will be performed. The purpose of these studies is to identify the mediators responsible for vaccine-derived immunity in vaccinated animals. To investigate their roles, blood will be fractionated from mice vaccinated with the protective dose determined by the studies described above. Immune serum (IS) samples will be obtained from mice at weeks 4 and 7, following the first and second vaccinations, respectively. Antibodies will be isolated for passive transfer. Further, antibodies will be compared against Ig-depleted serum. As a negative control, all experiments described will be performed with blood from unvaccinated mice.

### Example 3

### Production and Purification of Flavivirus VLPs Expressed in Yeast

This example describes a method of expressing flavivirus VLPs in Pichia pastoris and isolating the VLPs using mechanical lysis and sucrose gradient centrifugation. VLPs produced using this method sediment in a sucrose gradient in a similar manner to infectious virus particles

### VLP Production

Pichia pastoris glycerol stock was struck out on YEPD (1% yeast extract, 2% peptone, 2% dextrose) agar plate to isolate single colonies overnight. A single colony was grown in YEPD broth for 8 hours at 30° C. The yeast culture was diluted in minimal growth media (MGY: 1.3% YNB, 1.2% glycerol, 4×10⁻⁵% biotin) and grown for 16 hours. Protein expression was induced by dilution of the MGY culture into minimal methanol media (MM: 1.3% YNB, 4×10⁻⁵% biotin, 0.75% methanol). After 24 hours, yeast cells were mechanically lysed using Emulsiflex™ C-3. Yeast lysates were cleared of debris, and VLPs isolated by sucrose gradient centrifugation. Production of VLP proteins was verified by Western blot.

### Lysis Conditions

Five liters of Pichia pastoris culture was centrifuged to isolate cells. A one liter culture typically yields 2-4 grams of cell pellet. The pellet was thoroughly resuspended in ice-cold 10-25 mL PBS+1% BSA. The Emulsiflex™ C-3 lysis machine was primed with 50 mL PBS+1% BSA and resuspended yeast cell mixture was run through the machine 7-10 times with an average pressure of 20,000-25,000 psi. Lysis can be verified by light microscopy at 20× magnification. An aliquot of the lysate was removed and stored at −80° C. The remaining lysate was centrifuged at 10,000×g. The supernatant, referred to as S10-VLP, was removed to another tube, and the pellet was resuspended in PBS+1% BSA and stored at −80° C. An aliquot of S10-VLP was removed and stored at −80° C. The remaining S10-VLP was purified by sucrose gradient centrifugation (see below).

### Sucrose Cushion

Five mL 20% sucrose solution (20% sucrose in PBS+1 mM EDTA) was added to a SW28 ultracentrifuge tube. Approximately 15-20 mL S10-VLP lysate was layered on top of the sucrose cushion and ultracentrifuged at 25,000×g for 2 hours at 4° C. The supernatant was removed by slowly pouring off. The pellet was resuspended in PBS+1% BSA. VLP concentration was determined by Bradford Assay and VLP expression was determined by Western blot.

### Continuous Gradient

Two mL of a 10% sucrose solution (10% sucrose in PBS+1 mM EDTA) was added to a SW41 ultracentrifuge tube. With a clean pipette or Pasteur pipette, 2 mL 20% sucrose solution was delivered below the 10% solution. This step was repeated with 30-50% sucrose solutions, resulting in a 10 mL discontinuous sucrose gradient in the tube. The tube was allowed to sit in a cold room for 4 hours, resulting in a continuous gradient. S10-VLP was layered on top of the sucrose gradient and centrifuged in a SW41 swinging bucket rotor in the ultracentrifuge at 100,000×g for 16 hours at 4° C. The tubes were then removed from the buckets and pure VLPs appear as a white band in the bottom third of the tube. This band can be removed by inserting a needle and syringe directly into the band or by drip-fractionation through a hole in the bottom of tube. The sucrose was removed by dialysis in PBS or PBS+1% BSA. VLP concentration was determined by Bradford Assay and VLP expression was determined by Western blot.

### Results

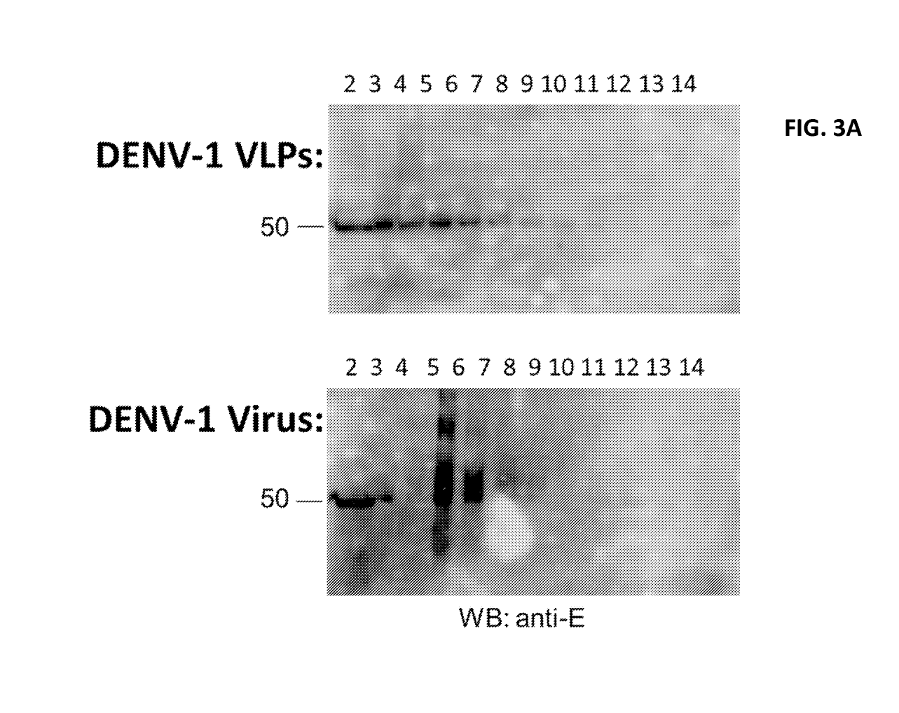

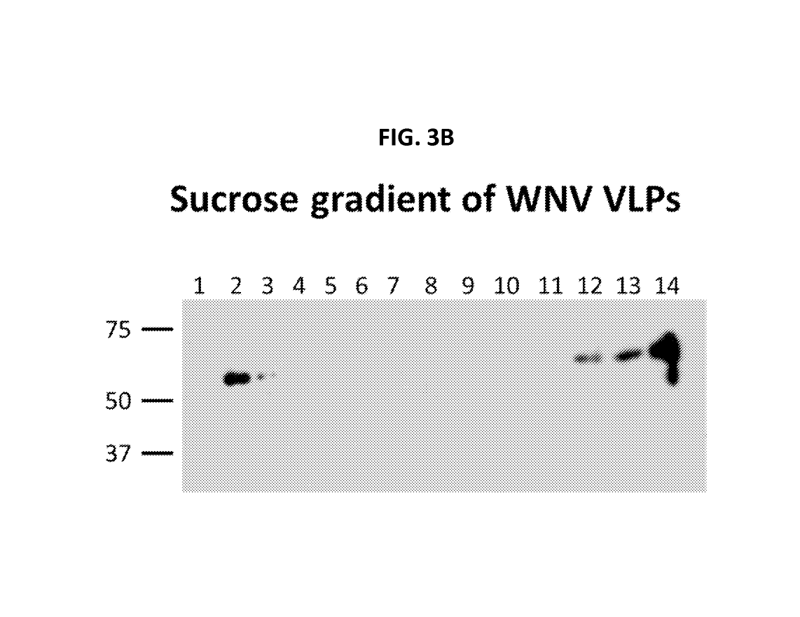

DENV-1 (Hawaii) and WNV (NY99) VLPs were produced in P. pastoris transformed with a vector (P. pastoris vector pPIC3.5) containing the C-prM-E coding region of each virus (SEQ ID NO: 1 for DENV-1 and SEQ ID NO: 9 for WNV). VLPs were isolated from 5 L of transformed yeast culture using mechanical lysis according to the procedure described above. As shown in FIGS. 3A and 3B, yeast-produced VLPs sediment in a sucrose gradient in a similar manner as infectious DENV-1 or WNV virus.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of producing flavivirus VLPs, comprising:
(a) transforming yeast cells with a nucleic acid molecule encoding a flavivirus premembrane (prM) protein; and at least one flavivirus envelope (E) protein or a flavivirus E protein lacking the transmembrane domain (EΔTM);
(b) cultivating the transformed yeast cells under conditions sufficient to allow for expression of the flavivirus proteins and formation of VLPs;
(c) subjecting the yeast cells to pressurized mechanical lysis; and
(d) isolating the VLPs from the yeast cell lysis.

2. The method of claim 1, wherein the nucleic acid molecule further encodes a flavivirus capsid (C) protein.

3. The method of claim 1, wherein the nucleic acid molecule further encodes at least one FMDV 2A autocatalytic site (2A) positioned between two flavivirus protein coding sequences.

4. The method of claim 1, wherein the nucleic acid molecule comprises the formula:
(a) C-prM-E;
(b) C-2A-prM-E;
(c) C-prM-2A-E;
(d) C-2A-prM-2A-E;
(e) C-prM-E₁-E₂-E₃-E₄;
(f) C-2A-prM-E₁-E₂-E₃-E₄;
(g) C-2A-prM-2A-E₁-E₂-E₃-E₄;
(h) C-prM-E₁-2A-E₂-2A-E₃-2A-E₄;
(i) C-2A-prM-E₁-2A-E₂-2A-E₃-2A-E₄;
(j) C-prM-2A-E₁-2A-E₂-2A-E₃-2A-E₄;
(k) C-2A-prM-2A-E₁-2A-E₂-2A-E₃-2A-E₄;
(l) C-prM-EΔTM;
(m) C-2A-prM-EΔTM;
(n) C-prM-2A-EΔTM; or
(o) C-2A-prM-2A-EΔTM.

5. The method of claim 1, wherein the flavivirus is selected from dengue virus type 1, dengue virus type 2, dengue virus type 3, dengue virus type 4, West Nile virus, Japanese encephalitis virus, yellow fever virus and St. Louis encephalitis virus.

6. The method of claim 4, wherein E₁, E₂, E₃ and E₄ are each from a different flavivirus.

7. (canceled)

8. The method of claim 1, wherein the nucleotide sequence of the nucleic acid molecule is codon-optimized for expression in yeast.

9-10. (canceled)

11. The method of claim 1, wherein mechanical lysis is carried out with an average pressure of about 20,000 to about 25,000 psi.

12. The method of claim 1, wherein isolating the VLPs from the yeast cell lysis comprises sucrose-gradient centrifugation.

13-16. (canceled)

17. An isolated nucleic acid molecule encoding a flavivirus capsid (C) protein; a flavivirus premembrane (prM) protein; and at least one flavivirus envelope (E) protein or a flavivirus E protein lacking the transmembrane domain (EΔTM), wherein the nucleic acid molecule further encodes at least one foot and mouth disease virus (FMDV) 2A autocatalytic site (2A) positioned between two flavivirus protein coding sequences, and wherein expression of the nucleic acid molecule in a host cell produces flavivirus virus-like particles (VLPs).

18. The nucleic acid molecule of claim 17 having the formula:
(a) C-2A-prM-E;
(b) C-prM-2A-E;
(c) C-2A-prM-2A-E;
(d) C-2A-prM-E₁-E₂-E₃-E₄;
(e) C-2A-prM-2A-E₁-E₂-E₃-E₄;
(f) C-prM-E₁-2A-E₂-2A-E₃-2A-E₄;
(g) C-2A-prM-E₁-2A-E₂-2A-E₃-2A-E₄;
(h) C-prM-2A-E₁-2A-E₂-2A-E₃-2A-E₄;
(i) C-2A-prM-2A-E₁-2A-E₂-2A-E₃-2A-E₄;
(j) C-2A-prM-EΔTM;
(k) C-prM-2A-EΔTM; or
(l) C-2A-prM-2A-EΔTM.

19. The nucleic acid molecule of claim 17, wherein the flavivirus is selected from dengue virus type 1, dengue virus type 2, dengue virus type 3, dengue virus type 4, West Nile virus, Japanese encephalitis virus, yellow fever virus and St. Louis encephalitis virus.

20. The nucleic acid molecule of claim 18, wherein E₁, E₂, E₃ and E₄ are each from a different flavivirus.

21. (canceled)

22. The nucleic acid molecule of claim 17, further encoding an immunomodulator.

23. The nucleic acid molecule of claim 22, wherein the immunomodulator comprises interleukin (IL)-1, IL-12, interferon (IFN)-α, IFN-β or IFN-γ.

24-25. (canceled)

26. The nucleic acid molecule of claim 17, wherein the nucleotide sequence of the nucleic acid molecule is codon-optimized for expression in yeast.

27. A vector comprising the nucleic acid molecule of claim 17.

28. The vector of claim 27, further comprising a promoter operably linked to the nucleic acid molecule encoding the flavivirus C, prM and E proteins.

29. The vector of claim 28, wherein the promoter is the phosphoglycerate kinase (PGK) promoter, glyceraldehyde-3-phosphate dehydrogenase (GAPDH) promoter, GAL1-10 promoter, methanol oxidase promoter, ADH1 promoter, CYC1 promoter, TDH3 promoter, or TEF promoter.

30. (canceled)

31. An isolated host cell comprising the vector of claim 27.

32. The host cell of claim 31, which is a yeast cell.

33-34. (canceled)

35. A VLP encoded by the nucleic acid molecule of claim 17.

36. A composition comprising the VLP of claim 35 and a pharmaceutically acceptable carrier.

37. (canceled)

38. A method of eliciting an immune response in a subject against flavivirus, comprising administering to the subject the VLP of claim 35.
